(19) United States
(12) Patent Application Publication
(10) Pub. No.: US 2018/0127722 A1
(43) Pub. Date: May 10, 2018

(54) COMPOSITION FOR TREATING TENDONITIS AND MANUFACTURE THEREOF

(71) Applicant: Gwo Xi Stem Cell Applied Technology Co., Ltd., Zhubei City (TW)

(72) Inventors: Shao-Chih Chiu, Zhubei City (TW); Wan-Sin Syu, Zhubei City (TW); Ming-Hsi Chuang, Zhubei City (TW); Po-Cheng Lin, Zhubei City (TW)

(21) Appl. No.: 15/416,502

(22) Filed: Jan. 26, 2017

(30) Foreign Application Priority Data
Nov. 4, 2016 (TW) 105135974

(51) Int. Cl. C12N 5/0775; A61K 35/28

(52) U.S. Cl. CPC C12N 5/0667; C12N 2500/30; A61K 35/28

(57) ABSTRACT

A composition for treatment of tendonitis is provided. The composition comprises a pretreated adipose derived stem cell (ADSC), wherein the ADSC is pretreated with butylidenephthalide, and the concentration of butylidenephthalide is greater or equal to. The composition of the invention has abilities to repair damaged tendon fiber, enhance tissue regeneration, and decrease inflammation. The invention also provides a method for manufacturing a composition for treatment of tendonitis, comprising culturing an ADSC in a medium containing butylidenephthalide.

### CROSS-REFERENCE TO RELATED APPLICATIONS

This Non-provisional application claims priority under 35 U.S.C. § 119(a) on Patent Application No(s). 105135974 filed in Taiwan, Republic of China Nov. 4, 2016, the entire contents of which are hereby incorporated by reference.

### FIELD OF THE INVENTION

The present invention relates to a pharmaceutical composition for treating tendonitis. In particular, the present invention relates to a pharmaceutical composition containing pre-treated adipose derived stem cells (ADSCs).

### BACKGROUND OF THE INVENTION

Tendons have been considered to transmit forces between muscles and bone; Otherwise, it can passively modulate forces during locomotion, providing additional stability with no active work. Not all tendons are required to perform the same functional role. For example, some tendons can store and recover energy at high efficiency.

Healthy tendons consist of parallel arrays of collagen fibers closely packed together. The dry mass of normal tendons is composed of about 86% collagen, 2% elastin, 1-5% proteoglycans, and 0.2% inorganic components. The collagen portion is made up of 95% type I collagen, with small amounts of other types of collagen. Three polypeptides coil to form tropocollagen. Many tropocollagens then bind together to form a fibril, and many of these then form a fiber. A bunch of collagen fibers forms a primary fiber bundle, and a group of primary fiber bundles forms a secondary fiber bundle. A group of secondary fiber bundles, in turn, forms a tertiary bundle, and the tertiary bundles make up the tendon.

Tendinopathy, the clinical presentation includes tenderness on redness and swelling and pain caused by overuse or extrinsic factors. Tendinopathy can be classified into “tendinitis” and “tendinosis”. Tendinitis refers to an acute tendon injury accompanied by great extent damage to tendons and inflammation. Tendinosis refers to a chronic tendon injury with degeneration at the cellular level caused by long-term overuse and no inflammation.

A rotator cuff tear could cause shoulder pain or discomfort in all population, particularly those age 65 years or older. Tendon injury causes collagen denaturation and disorder, increase of slime, angiogenesis, and loss of function in joint, and pain. The rotator cuff tear can be treated by surgery. However, the wound healing is poor after surgery.

For cell-based tissue regeneration, a potential advantage of using stem cells from an adult is that the patient's own cells can be expanded in culture and then reintroduced into the patient so that the cells would not be rejected by the immune system. However, the injured tendons cannot be completely treated by stem cells and the reason may be related to the growth factors secreted by human stem cells. Thus, it is necessary to find out a method for stimulating human adipose derived stem cells (hADSCs) and a growth factor for stimulating hADSCs to produce the repair-related growth factors relating to tendon repair.

It is therefore attempted by the applicant to deal with the above situation encountered in the prior art.

### SUMMARY OF THE INVENTION

Butylidenephthalide, a plant extract extracted from Angelica sinensis. Angelica sinensis has been considered to have the effect of clearing bold and promoting blood circulation, and usually used as a drug for treating anemia, disturbance of menstruation, and constipation.

In view of the above-mentioned problem, the present invention provides a pharmaceutical composition for treating tendonitis comprising a pre-treated adipose derived stem cell (ADSC).

Furthermore, the present invention provides a method for treating tendonitis in a subject, wherein the method comprising an administering to said subject a pharmaceutical composition, comprising an adipose derived stem cell pre-treated by butylidenephthalide.

In one embodiment, the tendonitis includes an infraspinatus tendinitis and/or a supraspinatus tendinitis.

In one embodiment, the infraspinatus tendinitis means the infraspinatus tendon and muscle middle is suffer from inflammation.

In one embodiment, the infraspinatus tendinitis means the infraspinatus tendon is suffer from tenocyte structure damage.

In one embodiment, the infraspinatus tendinitis means the infraspinatus muscle is suffer from inflammation.

In one embodiment, the supraspinatus tendinitis means the supraspinatus muscle is suffer from inflammation.

In one embodiment, the supraspinatus tendinitis means the supraspinatus tendon is suffer from fiber arrangement.

In one embodiment, the supraspinatus tendinitis means the supraspinatus tendon and muscle middle is suffer from inflammation.

In one embodiment, the administering is via injection.

In one embodiment, the injection is on day 3.

In one embodiment, the pre-treated adipose derived stem cell express the SCX, DCN, TNC or COL1A1 marker on the cell.

In one embodiment, the protein secreted from the pre-treated hADSC is COL1.

In one embodiment, the pre-treated hADSC is pretreated with butylidenephthalide.

In one embodiment, the butylidenephthalide has a concentration of 2.5 to 5 μg/ml.

In one embodiment, the number of the pre-treated hADSC is 1×10⁵ to 3×10⁸ cells/ml.

The present invention also provides a method of manufacturing a pharmaceutical composition for treating tendonitis, comprising pre-treating an hADSC.

In one embodiment, the pre-treating step comprises culturing the hADSC in a medium containing butylidenephthalide.

In one embodiment, the hADSC is cultured in a medium containing butylidenephthalide for 96 to 168 hours.

In one embodiment, the butylidenephthalide has a concentration of 2.5 to 5 μg/ml.

Detailed description of the invention is given in the following embodiments with reference to the accompanying drawings.

### BRIEF DESCRIPTION OF THE DRAWINGS

FIG. 1 illustrates the effect of butylidenephthalide on the activity of hADSC

FIG. 2 illustrates a schematic of the animal experiments according to the present invention.

FIG. 3 illustrates the appearance of infraspinatus tendon on day 7, 14, 21, and 28 after induction of inflammation by type II collagenase. The infraspinatus tendon is a normal tendon on day 0.

FIG. 4 illustrates histological sections (Hematoxylin-Eosin stain) of infraspinatus tendon on day 7, 14, 21, and 28 after induction of inflammation in the treated and untreated (vehicle) groups.

FIG. 5 illustrates the appearance of supraspinatus tendon on day 7, 14, 21, and 28 after induction of inflammation by type II collagenase. The supraspinatus tendon is a normal tendon on day 0.

FIG. 6 illustrates histological sections (Hematoxylin-Eosin stain) of supraspinatus tendons on day 7, 14, 21, and 28 after induction of inflammation in the treated and untreated (vehicle) groups.

FIG. 7 illustrates a graph showing the tensile ability of infraspinatus tendons treated by hADSC on day 3, 7, 14, 21, and 28 after induction of inflammation by type II collagenase. On day 0, non-treatment is considered as the control group (normal tendon).

FIG. 8 illustrates a graph showing the tensile ability of supraspinatus tendons treated by hADSC on day 3, 7, 14, 21, and 28 after induction of inflammation by type II collagenase. On day 0, non-treatment is considered as the control group (normal tendon).

FIG. 9 illustrates the appearance of infraspinatus tendon injected with butylidenephthalide pretreated hADSC on day 3, 7, 14, 21, and 28 after induction of inflammation by type II collagenase. The infraspinatus tendon is a normal tendon on day 0.

FIG. 10 illustrates histological sections (Hematoxylin-Eosin stain) of infraspinatus tendons on day 7, 14, 21, and 28 after induction of inflammation in the untreated (vehicle) and treated (butylidenephthalide) groups.

FIG. 11 illustrates the appearance of supraspinatus tendons injected with butylidenephthalide pretreated hADSC on day 7, 14, 21, and 28 after induction of inflammation by type II collagenase. The supraspinatus tendon is a normal tendon on day 0.

FIG. 12 illustrates histological sections (Hematoxylin-Eosin stain) of supraspinatus tendons on day 7, 14, 21, and 28 after induction of inflammation in the untreated (vehicle) and treated (butylidenephthalide pretreatment) groups.

FIG. 13 illustrates the histological sections (Elastic stain) of the collagen in supraspinatus tendons. The collagen is significantly reduced in supraspinatus tendons treated with type II collagenase. By comparison, more collagens are observed in the supraspinatus tendons injected with butylidenephthalide (2.5 μ/ml) pretreated hADSC.

FIG. 14 illustrates a graph showing the tensile ability of infraspinatus tendons treated with pre-treated hADSC (butylidenephthalide pretreatment) on day 3, 7, 14, 21, and 28 after induction of inflammation by type II collagenase. On day 0, non-treatment is considered as the control group (normal tendon).

FIG. 15 illustrates a graph showing the tensile ability of supraspinatus tendons treated by pre-treated hADSC (butylidenephthalide pretreatment) on day 3, 7, 14, 21, and 28 after induction of inflammation by type II collagenase. On day 0, non-treatment is considered as the control group (normal tendon).

FIG. 16 illustrates a graph showing the ultimate load failure of tendons on day 7 and 14 after injection of hADSC (2.5 μg/ml butylidenephthalide pretreatment) with different cell densities (2×10⁴ or 1×10⁵ cell/ml).

FIG. 17 illustrates a graph showing a significant increase of COL1A1 and SCX mRNA expression in hADSC at high cell density (1×10⁵ cell/ml) after 96 and 168 hours of butylidenephthalide (2.5 μg/ml) treatment.

FIG. 18 illustrates a graph showing a significant increase of COL1A1 protien expression in hADSC at high cell density (1×10⁵ cell/ml) after 96 and 168 hours of butylidenephthalide (2.5 μg/ml) treatment.

### DETAILED DESCRIPTION OF THE INVENTION

The present invention provides a pharmaceutical composition for treating tendonitis, comprising a pre-treated stem cell.

The term “stem cell” is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or symmetric replication, where the two daughter cells after division can have different phenotypes; extensive self-renewal capacity; capacity for existence in a mitotically quiescent form; and clonal regeneration of all of the tissue, for example the ability of hematopoietic stem cells to reconstitute all hematopoietic lineages. The stem cell of the invention includes, but are not limited to, a blood stem cell, an adipose stem cell, a bone marrow mesenchymal stem cell, a mesenchymal stem cell, a neural stem cell, a skin stem cell, an embryonic stem cell, an endothelial stem cell, a hepatic stem cell, a pancreatic stem cell, an intestinal epithelium stem cell, or a germ stem cell, preferably adipose derived stem cell (ADSC).

The ADSCs of the invention have the capacity to differentiate into mesodermal tissues, such as mature adipose tissue, bone, various tissues of the heart (e.g., pericardium, epicardium, epimyocardium, myocardium, pericardium, valve tissue, etc.), dermal connective tissue, hemangial tissues (e.g., corpuscles, endocardium, vascular epithelium, etc.), hematopeotic tissue, muscle tissues (including skeletal muscles, cardiac muscles, smooth muscles, etc.), urogenital tissues (e.g., kidney, pronephros, meta- and meso-nephric ducts, metanephric diverticulum, ureters, renal pelvis, collecting tubules, epithelium of the female reproductive structures, mesodermal glandular tissues, and stromal tissues (e.g., bone marrow).

The ADSCs of the invention are isolated from adipose tissue. The adipose tissue can be obtained from an animal by any suitable method. A first step in any such method requires the isolation of the adipose tissue from the source animal. The animal can be alive or dead, so long as adipose stromal cells within the animal are viable. Typically, human adipose tissue is obtained from a living donor, using well-recognized protocols such as surgical or suction lipectomy. The preferred method to obtain human adipose tissue is by excision or liposuction procedures well known in the art. Preferably, the inventive ADSCs are isolated from a liposuction aspirate.

In one embodiment, the gene expression of ADSC is selected from SCX, DCN, TNC, or COL1A1, preferably COL1.

It shall be noted that the adipose stem cell of the present invention is treated with butylidenephthalide.

In the present invention, the number of the adipose stem cell is 1×10⁵ to 3×10⁸ cell/ml.

The present invention further provide a method of manufacturing a composition for treating tendonitis, comprising pre-treating an adipose stem cell.

In one embodiment, the adipose stem cell is pretreated with butylidenephthalide. Preferably, the adipose stem cell is cultured in a medium containing butylidenephthalide.

The basal medium that is used in the present invention is a conventional basal medium that is known to be suitable for the culture of stem cells in the art. Examples of the basal medium that is used in the present invention include DMEM, MEM, K-SFM media and the like.

In one particular embodiment, the medium of the present invention may be a DMEM containing 10% fetal bovine serum, 1% L-glutamine acid, 1% nonessential amino acids (NEAA), 1% sodium pyruvate, and 2.5 μg/ml butylidenephthalide. The concentration of butylidenephthalide may be 2.5 to 5 μg/ml.

The stem cell may be cultured in a medium containing butylidenephthalide for 96 to 168 hours.

The pharmaceutical composition of the present invention may be used to effectively treat tendonitis in a subject for arranging the collagen fibers in tendons, changing the shape of tendon cells to oblate, enhancing tendons healing. The pharmaceutical composition may stimulate the self-repair capacity of tendons to increase its tensile strength.

The composition of the present invention comprises an effective amount of a pre-treated adipose stem cell, which may be administrated to a subject by a necessary procedure. The composition can be administered subcutaneously, intramuscularly, or intraperitoneally.

Additional specific embodiments of the present invention include, but are not limited to the following:

### EXAMPLE 1

### Treatment of Rotator Cuff Tendinitis Using hADSC

a. Material and Method

i. Stem Cells Culture

Human adipose derived cells (hADSCs) were cultured in DMEM medium containing 10% fetal bovine serum, 1% L-glutamine acid, 1% nonessential amino acids (NEAA), and 1% sodium pyruvate.

ii. Cell Activity

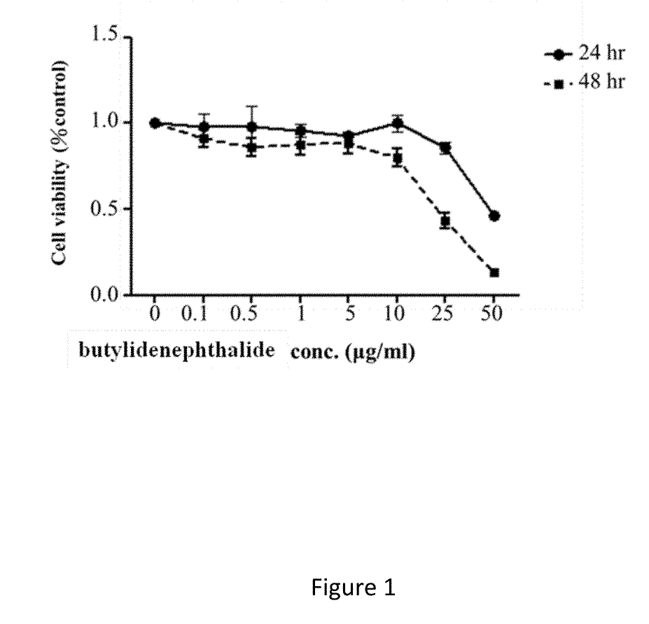

hADSCs were seeded on a 96-well plate at a density of 3×10³ cell/100 μl, and the 96-well plate was incubated at 37° C. in a humidified 5% CO₂ atmosphere. After 16 hours, butylidenephthalide of various concentrations was added to the 96-well plate and then incubated at 37° C. in a 5% CO₂ incubator. After 24 and 48 hours, the cell viability was determined. In cell viability assay, 100 μl of 10% MTT reagent was added to each well and then incubated in an incubator for 2 hours. 150 μl DMSO was added and mixed for 15 minutes. The absorbance (OD) of the aqueous solution was determined at 570 nm. According to FIG. 1, butylidenephthalide would not affect the cell activity if its concentration was blow 5 μg/ml. Therefore, 2.5 μg/ml of butylidenephthalide was selected to be used in the following experiments.

iii. Culture of Pre-Treated hADSC In Vitro

20 ml of 1.0×10⁵ or 0.2×10⁵ cells/ml hADSCs in DMEM medium (10% fetal bovine serum, 1% L-glutamine, 1% non-essential amino acid (NEAA), and 1% sodium pyruvate) was placed on a 15-cm dish, mixed with 2.5 μg/ml butylidenephthalide, and then incubated at 37° C. in a 5% CO₂ incubator for 96 hours. After the medium was removed and replaced with fresh medium, hADSCs was cultured under the same condition as described above until 168 hours.

iv. Animal Experiments

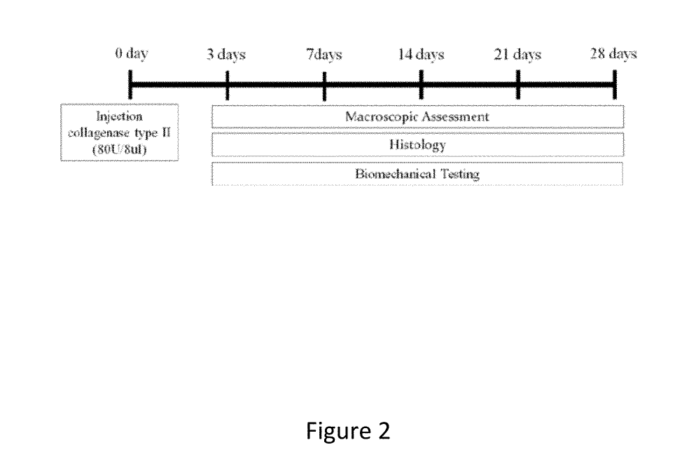

Spregue-Dawley (SD) female mice, weighing 250-300 g (12-13 weeks-old) were obtained from National Laboratory Animal Center. The mice were anesthetized by 0.01 μl/g of cholral hydrate for the animal test. Type II collagenase was injected to supraspinatus tendon between coracoids and clavicle of mice by using inserting the 27 G needles through skin at 45 degrees at a flow rate of 80 U/8 μl/min. After injection, the mice rested for three days. The ADSCs were pre-treated with butylidenephthalide, moved to 50-ml tube from 15-cm dish, and then centrifuged by 800 rpm for 3 minutes to remove supernatant. The pellets were suspended with PBS to obtain 6×10⁶ cells/ml of the pre-treated hADSCs. Next, the hADSCs were added to 1.5-ml tube and centrifuged by 800 rpm for 3 minutes to remove supernatant. The pellets were re-dissolved in 20 μl PBS. 3×10⁶ cells/10 μl pre-treated hADSCs were topically injected to the supraspinatus tendons between coracoids and clavicle by microinjector. The suprspinatus tendons were analyzed on day 3, 7, 14, 21, 28 after injection (FIG. 2).

b. Results

i. Appearance of Infraspinatus Tendon

The appearance of infraspinatus tendon was determined on day 3, 7, 14, 21, and 28 after treatment of type II collagenase. The untreated infraspinatus tendon on day 0 was considered as a control group (normal tendon).

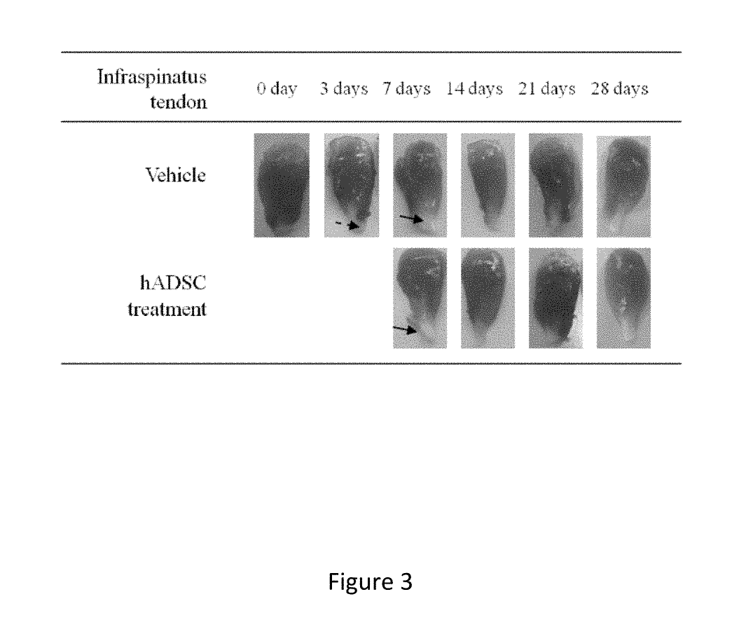

Referring to FIG. 3, for treated group, the slight bleeding and swelling were observed on muscles and tendons compared to control group (normal tendon) on day 3. On day 7, the tendons had new whitish translucent tissues (new tissue growth) in both treated and untreated groups. On day 14 and 21, the color of new tissues was changed to opaque from translucent. On day 28, the appearance of tendons treated and untreated with hADSCs was same as that of the normal tendon.

ii. Histological Section of Infraspinatus Tendon

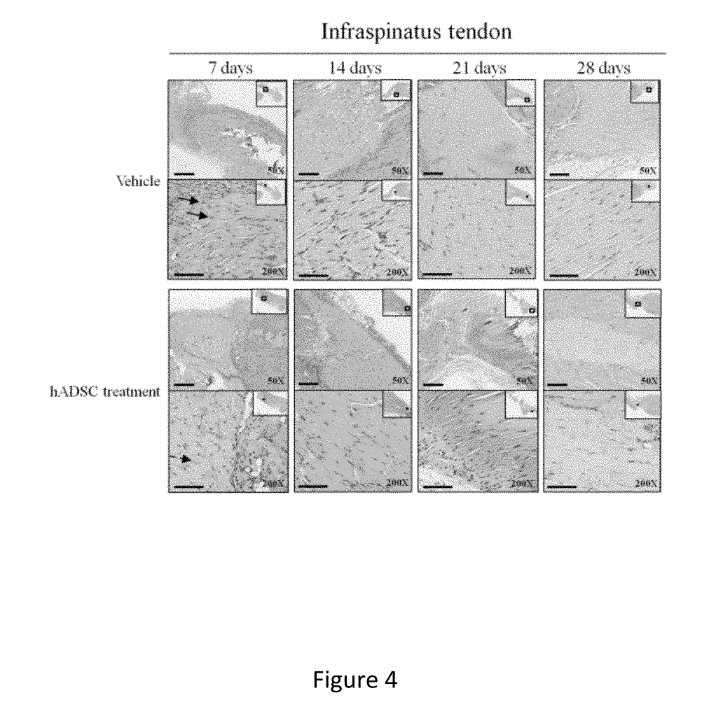

3 days after injection of hADSCs, an analysis of histological sections was carried out. In FIG. 4, the untreated tendons were ruptured resulted in the aggregation of inflammatory cells and a change in tendon cell morphology on day 7. However, some tendons were repaired, tendon cells and collagens restored smoothness, and inflammatory cells were decreased significantly after hADSCs injection. On day 14, the tendons were smoother compared to that on day 7 and the morphology of tendon cells was changed to strip-shape. The inflammation were also decreased as the time has passed. On day 21 and 28, the inflammatory cells were not observed, the shape of the tendon cells was changed to oblate, and collagen fibers were well oriented and ran parallel in the tendons for treated group. The appearance of tendons in treated or untreated groups on day 21 and 28 was same as that of normal tendons.

Additionally, the inflammation of tendons was determined by histological section images. The histological sections were classified into three regions including tendon, tendon and muscle middle, and muscle to evaluate the aggregation of inflammatory cells, morphology of tendon cells, and arrangement of tendon fibers. Based on the aggregation of inflammatory cells, morphology of tendon cells, and arrangement of tendon fibers, the degree of damage was classified into five grades (abnormal level): “−”: 0%, “−/+”: 17%, “+”: 34%, “+/++”: 50%, “++”: 66%, “++/+++”: 82%, and “+++”: 100%. For treated group, the shape of inflammatory cells and tendon cells were more oblate, and collagen fibers were more regular and uniform compared to untreated group as shown in Table 1.

iii. Appearance of Supraspinatus Tendon

The appearance of supraspinatus tendon was observed on day 3, 7, 14, 21, and 28 after treatment of type II collagenase. The untreated supraspinatus tendon on day 0 was considered as a control group (normal tendon).

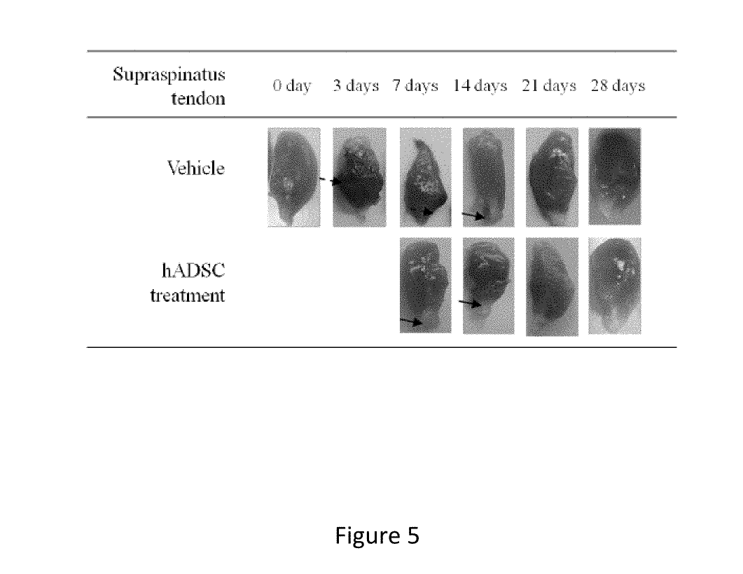

Referring to FIG. 5, compared to control group (normal tendon), serious muscle bleeding and swelling and tendon rupture were observed on day 3. For the untreated group, slight bleeding at the edge of muscles was observed and the front tip of the tendon was coated by whitish translucent tissues on day 7. The bleeding and swelling of tendons apparently decrease compared to the treated group. The swelling was improved on day 7 compared to that on day 3.

On day 14, the whitish translucent tissues also were observed on the periphery of the tendons in untreated group. However, the color of tissues coated on the periphery of the tendons, was changed to opaque. On day 21, the whitish translucent tissues were gradually changed to opaque tissues in the untreated group, and the whitish translucent tissues were disappeared in the treated group. On day 28, the appearance of tendons was restored to normal in both treated and untreated groups, and the appearance of tendons were same as that in the control group.

iv. Histological Section of Supraspinatus Tendon

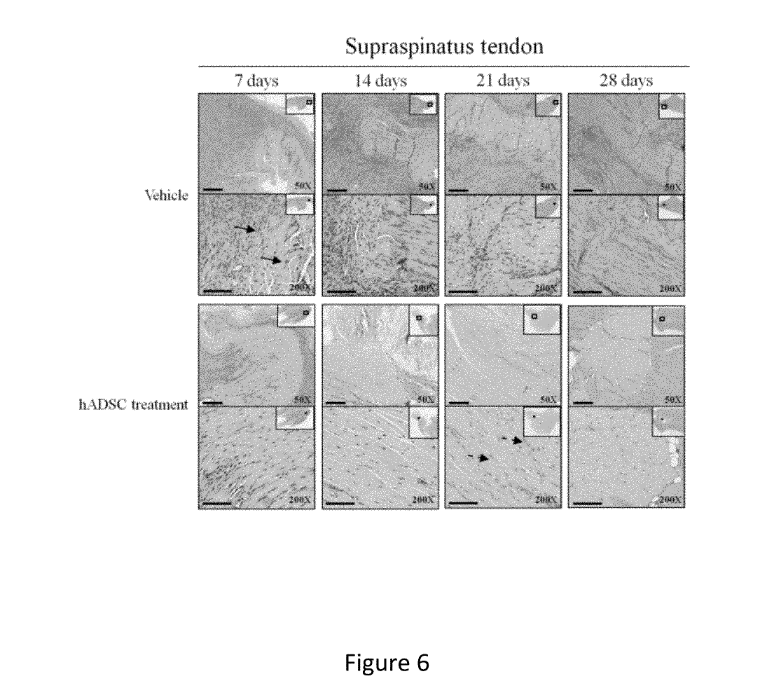

3 days after injection of hADSCs, an analysis of histological sections was carried out. In FIG. 6, for untreated group, the informatory cells were surrounded around the tendons and muscles, and the tendon rupture resulted in structural abnormalities of collagen fibers on day 7. After hADSCs treatment, the tendon fibers were apparently repaired and collagen fibers were smoother compared to the untreated group. However, a small number of inflammatory cells were still aggregated on the tendons. For treated group, the collagen fibers were smooth, aggregated inflammatory cells apparently decrease, and the shape of the tendon cells was changed to oblate on day 14. On day 21 and 28, inflammatory cells were not observed in both untreated and treated groups, the shape of tendon cells was restored to oblate (normal shape), and the collagen fibers were well oriented and ran parallel.

Additionally, the inflammation of tendons was determined by histological section images. The histological sections were classified into three regions including tendon, tendon and muscle middle, and muscle to evaluate the aggregation of inflammatory cells, morphology of tendon cells, and arrangement of tendon fibers. Based on the aggregation of inflammatory cells, morphology of tendon cells, and arrangement of tendon fibers, the degree of damage was classified into five grades (abnormal level), “−”: 0%, “−/+”: 17%, “+”: 34%, “+/++”: 50%, “++”: 66%, “++/+++”: 82%, and “+++”: 100%. Compared to the untreated group, the shape of inflammatory cells and tendon cells was more oblate, and collagen fibers were smoother as shown in Table 2.

### EXAMPLE 2

### Recovery of Tendon Strength in Mice

a. Material and Method

i. Biomechanical Testing

The mice were sacrificed with an overdose of chloral hydrate (i.p.) to obtain the infraspinatus tendon or supraspinatus tendon with humerus. The tendons were wrapped in gauze saturated with PBS, and then wrapped in aluminum foil for temporary storage. Before the test process started, the humerus was placed on an acrylic mold fixed by rubber bands, and the muscles on the end of the tendons were pierced by paper clips. The acrylic mold then was placed on a material-testing system (JSVH1000, Japan Instrumentation System, Nara, Japan) to fix the muscles using freeze spray (−60° C.). A tensile test was performed by elongating the tendon at a rate of 10 mm/min until tendon rupture to determine the ultimate load failure of the tendons.

b. Results

ii. Biomechanical Test of Infraspinatus Tendon

The tensile strength of infraspinatus tendon was determined on the different days in the treated and untreated groups by the method as described above.

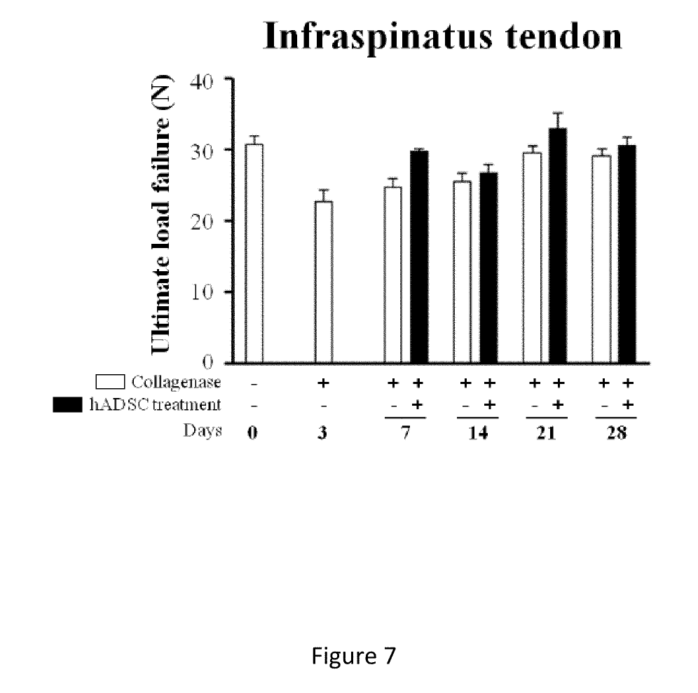

Referring to FIG. 7, the tensile strength of the infraspinatus tendon on day 3, 7, 14, 21, and 28 after treatment of type II collagenase was 22.79±3.85N, 24.83±3.07N, 25.50±3.03N, 29.52±2.67N, and 28.95±3.46N separately. The tensile strength of normal tendon was 30.83±2.77N.

3 days after treatment of type II collagenase, hADSCs were injected by the same method. On day 7, the tensile strength of the tendons injected with hADSCs was 30.57±2.12N, which was 5.74N higher than that of the tendons without hADSCs treatment. On day 14, the tensile strength of the tendons injected with hADSCs was 26.07±2.76N, which was 0.57N higher than that of the tendons without hADSCs treatment. On day 21, the tensile strength of the tendons injected with hADSCs was 8.5N higher than that of the tendons of untreated group. On day 28, the tensile strength of the tendons injected with hADSCs was 30.99±3.88N, which was higher than that of the untreated group.

iii. Biomechanical Test of Supraspinatus Tendon

The tensile strength of supraspinatus tendon was determined on the different days in the treated or untreated groups by the method as described above.

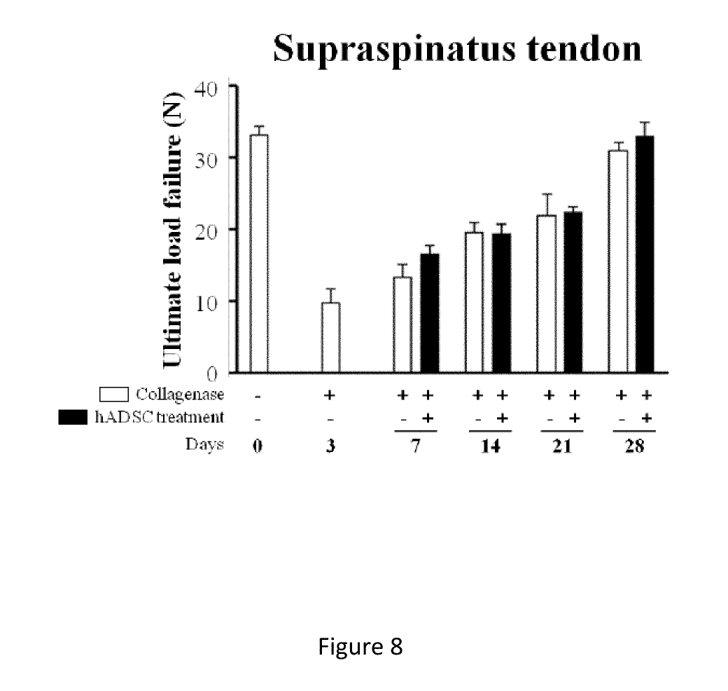

In FIG. 8, the tensile strength of the supraspinatus tendon on day 3, 7, 14, 21, and 28 after treatment of type II collagenase was 9.71±6.6N, 13.26±4.34N, 19.46±3.59N, 21.99±7.39N, and 30.88±3.68N, separately. The tensile strength of the normal tendon (day 0) was 33.11±2.78N. After 3 days of type II collagenase treatment, hADSCs were injected by the same method. On day 7, the tensile strength of the tendons injected with hADSCs was 15.24±4.29N, which was higher than that of the tendons of the untreated group. On day 14, the tensile strength of the tendons injected with hADSCs was 19.36±3.19N, which was lower than that of the tendons of the untreated group. On day 21, the tensile strength of the tendons injected with hADSCs was 22.41±1.76N, which was higher than that in the tendons of untreated group. On day 28, the tensile strength of the tendons injected with hADSCs was higher than that of the tendons in the untreated group.

### EXAMPLE 3

### Effect of Butylidenephthalide on Acute Tendonitis Treated by hADSC

a. Material and Method

i. Pretreatment of Stem Cells

hADSCs were pre-treated with butylidenephthalide. The hADSCs were cultured in DMEM medium containing 10% fetal bovine serum, 1% L-glutamine acid, 1% nonessential amino acids (NEAA), 1% sodium pyruvate, and 2.5 μg/ml butylidenephthalide. The same procedure carried out in Example 1 was repeated to perform animal experiments.

b. Results

i. Appearance of Infraspinatus Tendon

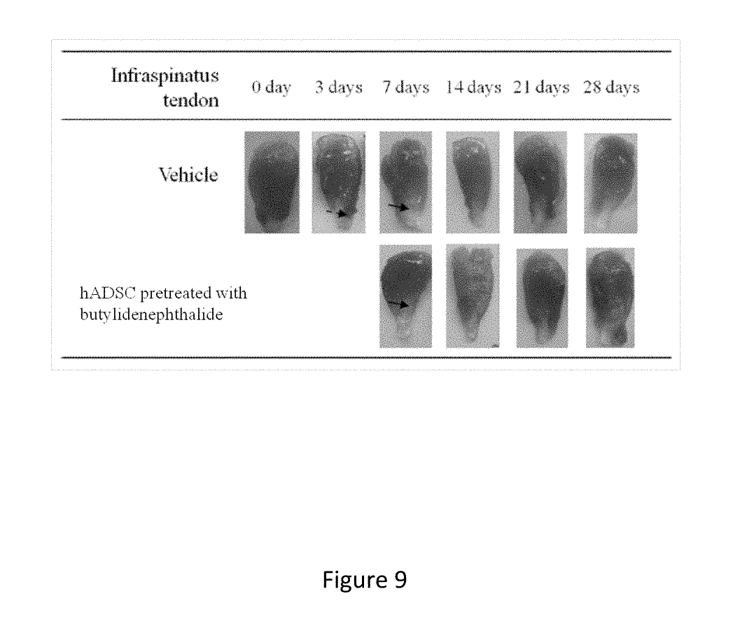

Referring to FIG. 9, slight bleeding and swelling at the edge of the infraspinatus tendons and muscles were observed on day 3 after treatment of type II collagenase. On day 7, the new whitish translucent tissues, so-called “new tissues” were observed on the tendons in the untreated and treated groups. On day 14, the tissues on front tip of the tendons were changed to whitish opaque from whitish translucent. On day 21, the tissues on rear tip of the tendons also were changed to whitish opaque from whitish translucent. On day 28, the appearance of tendons treated or untreated with hADSCs was same as that of normal tendons.

ii. Histological Section of Infraspinatus Tendon

3 days after injection of tendon inflammation, hADSCs treated with butylidenephthalide were topically injected to supraspinatus tendon between coracoids and clavicle. After 4 days of injection, the histological sections of suprspinatus tendons were analyzed.

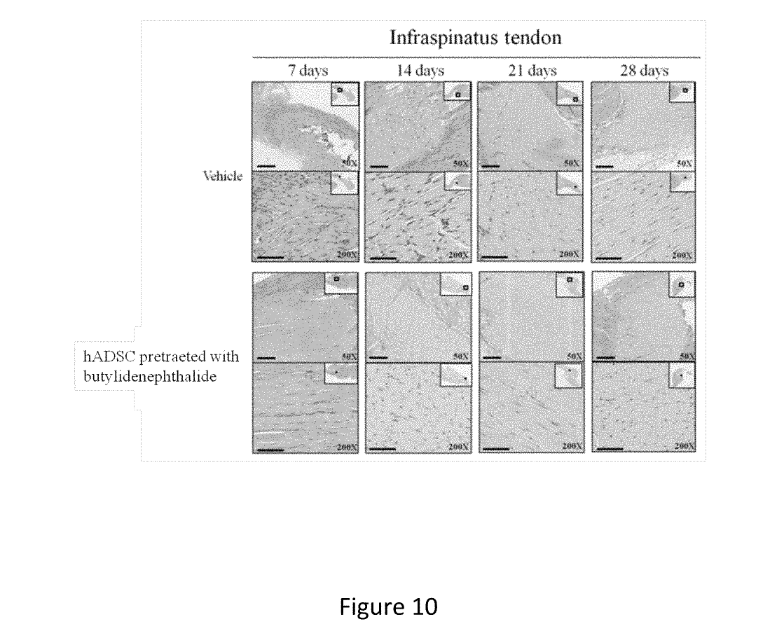

Referring to FIG. 10, for the untreated group, the tendons were ruptured by type II collagenase resulted in aggregation of inflammatory cells and a change in tendon cell morphology on day 7. However, some tendons were repaired, arrangement of tendon cells and collagens restored smoothness, and inflammatory cells were decreased significantly after injection of pre-treated hADSCs. On day 14, the tendons were smoother compared to the untreated group, the morphology of tendon cells was changed to strip-shape, and the aggregation of the inflammation cells was not observed. On day 21 and 28, the inflammatory cells were not observed in tendons, the shape of the tendon cells was changed to oblate, and collagen fibers were well oriented and ran parallel in the tendons for the untreated group. On day 21 and 28, the appearance of tendons treated or untreated with hADSCs was same as that of normal tendons.

Additionally, the inflammation of tendons was determined by histological section images. The histological sections were classified into three regions including tendon, tendon and muscle middle, and muscle to evaluate the aggregation of inflammatory cells, morphology of tendon cells, and arrangement of tendon fibers. Based on the aggregation of inflammatory cells, morphology of tendon cells, and arrangement of tendon fibers, the degree of damage was classified into five grades, “−”: 0%, “−/+”: 17%, “+”: 34%, “+/++”: 50%, “++”: 66%, “++/+++”: 82%, and “+++”: 100%. Compared to the untreated group, the shape of inflammatory cells and tendon cells were more oblate, and the collagen fibers were smoother as shown in Table 3.

iii. Appearance of Supraspinatus Tendon

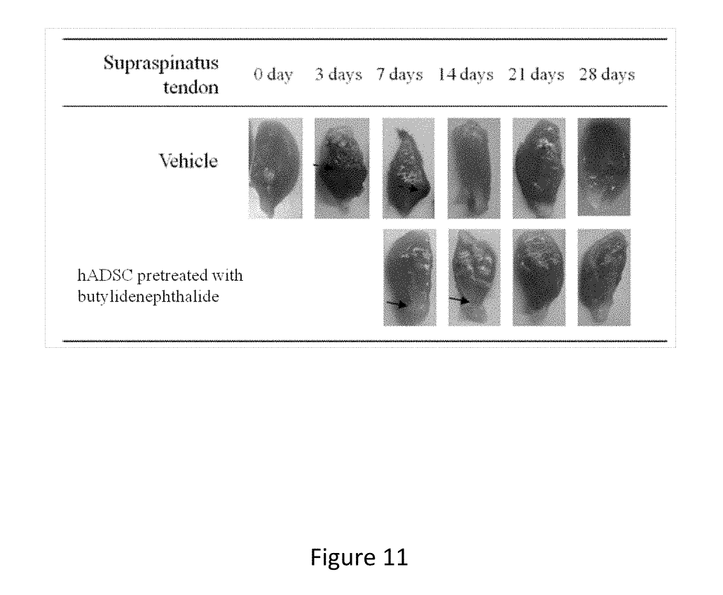

Referring to FIG. 11, the muscle bleeding and swelling and tendon rupture were observed on day 3 after treatment of type II collagenase. For the untreated group, the bleeding and swelling were significantly improved, the treated tendons were coated by a layer of whitish translucent tissues, so-called new tissues and the muscle bleeding and swelling were disappeared on day 7. On day 14, the new tissues were also observed on the edge of tendon in the untreated tendon, and the whitish translucent tissues were still located on the front tip of treated tendons. On day 21, the whitish translucent tissues at the front tip of tendons were changed to opaque tissues in the treated and untreated groups. On day 28, the appearance of tendons in the treated and untreated groups was same as that in the control group (normal tendon).

iv. Histological Section of Supraspinatus Tendon

After 3 days of inflammation induction, the hADSCs pre-treated with butylidenephthalide were topically injected to supraspinatus tendon between coracoids and clavicle. After 4 days of injection, the histological sections of suprspinatus tendons were analyzed.

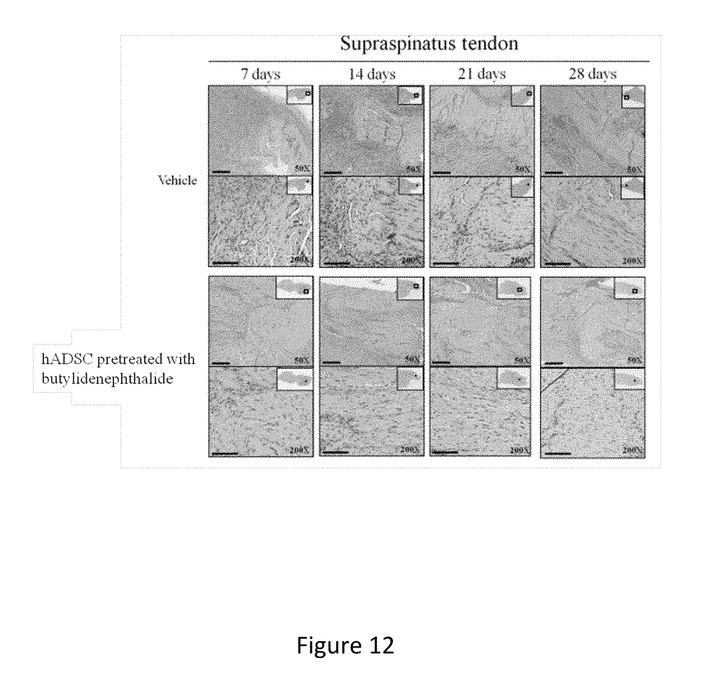

Referring to FIG. 12, for the untreated group, the tendons were ruptured, arrangement of collagen fibers was irregular, and inflammatory cells were aggregated on day 7. In contrast, the collagen fibers of tendons were smooth/regular, and inflammatory cells were significantly decreased in the treated group. In the untreated group, the injured tendons had a self-repair capacity on day 14 compared to that on day 7. In the treated group, the collagen fibers of the tendons were smoother on day 14 compared to that of tendon on day 7, the morphology of the tendons was changed to oblate, and the inflammatory cells were decreased. On day 21 and 28, the inflammatory cells were not observed, and collagen fibers were well oriented and ran parallel in the tendons in the treated and untreated groups.

v. Elastic Fiber Stain of Supraspinatus Tendon

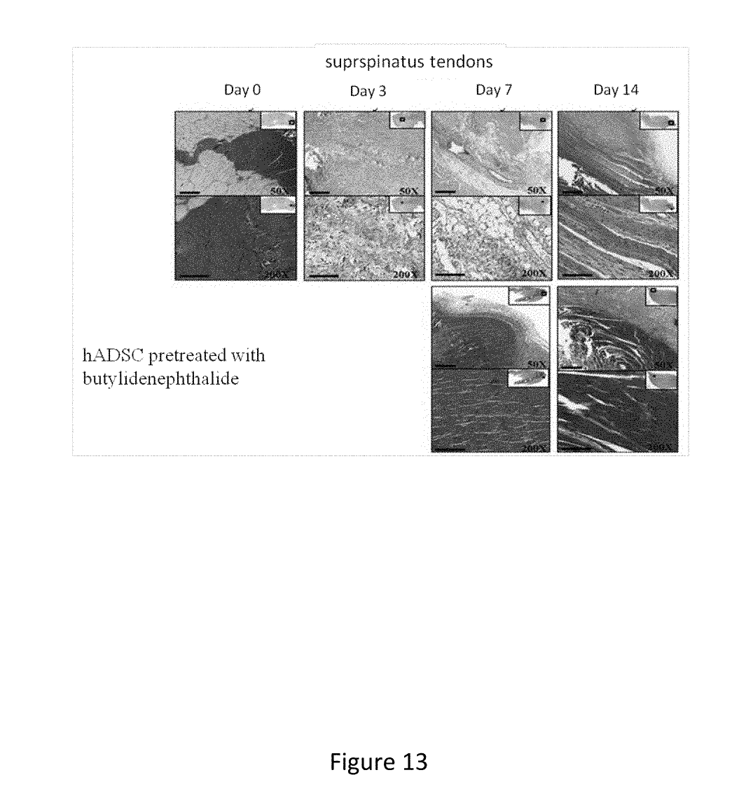

FIG. 13 shows the histological sections (Elastic stain) of collagen of the supraspinatus tendons. The collagen of tendons was apparently decreased because the injection of collagenase. In contrast, after the tendons were treated with butylidenephthalide (2.5 μg/ml) pretreated hADSC, the tendons had more collagens.

Additionally, the inflammation of tendons was determined by histological section images. The histological sections were classified into three regions including tendon, tendon and muscle middle, and muscle to evaluate the aggregation of inflammatory cells, morphology of tendon cells, and arrangement of tendon fibers. Based on the aggregation of inflammatory cells, morphology of tendon cells, and arrangement of tendon fibers, the degree of damage was classified into five grades, “−”: 0%, “−/+”: 17%, “+”: 34%, “+/++”: 50%, “++”: 66%, “++/+++”: 82%, and “+++”: 100%. Compared to untreated group, the shape of inflammatory cells and tendon cells were more oblate, and collagen fibers were smoother as shown in Table 4.

vi. Biomechanical Test of Infraspinatus Tendon

The tensile strength of tendons was determined in control, treated, and untreated groups by the same method as described above.

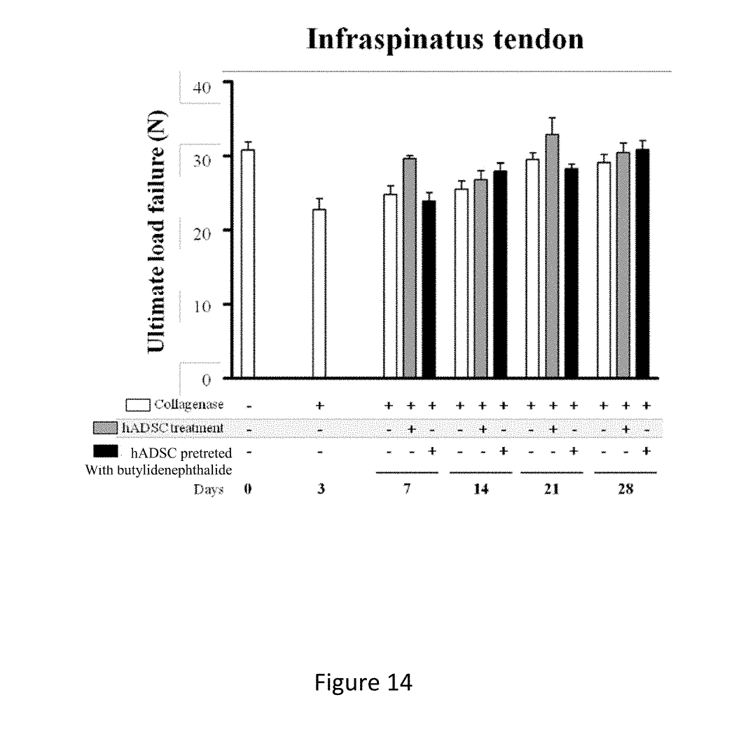

Referring to FIG. 14, after treatment of type II collagenase, the tensile strength of infraspinatus tendons on day 3, 7, 14, 21, and 28 was 22.79±3.85N, 24.83±3.07N, 25.50±3.03N, 29.52±2.67N, and 9.11±3.29, separately. The tensile strength of the normal tendon (day 0) was 30.83±2.77N.

On day 3 after injection of collagenase, the hADSCs and pre-treated hADSCs were injected to mice, respectively. On day 7, the tensile strength of the tendons treated with hADSCs was higher than that of untreated tendons, and the tensile strength of tendons treated with butylidenephthalide pretreated hADSCs (pretreated hADSCs) was same as that of untreated tendons. On day 14, the tensile strength of tendons treated with hADSCs and pretreated hADSCs was 26.07±2.76N and 28.64±2.81N, respectively. The tensile strength of these two groups was higher than that of untreated tendons.

On day 21, the tensile strength of tendons treated with hADSCs was higher, but the tensile strength of tendon treated with pretreated hADSCs was lower compared to untreated tendons. On day 28, the tensile strength of tendons treated with hADSCs and pre-treated hADSCs was 30.99±3.88N and 30.03±3.16N, respectively. The tensile strength of these two groups was higher than that of untreated tendons.

vii. Biomechanical Test of Supraspinatus Tendon

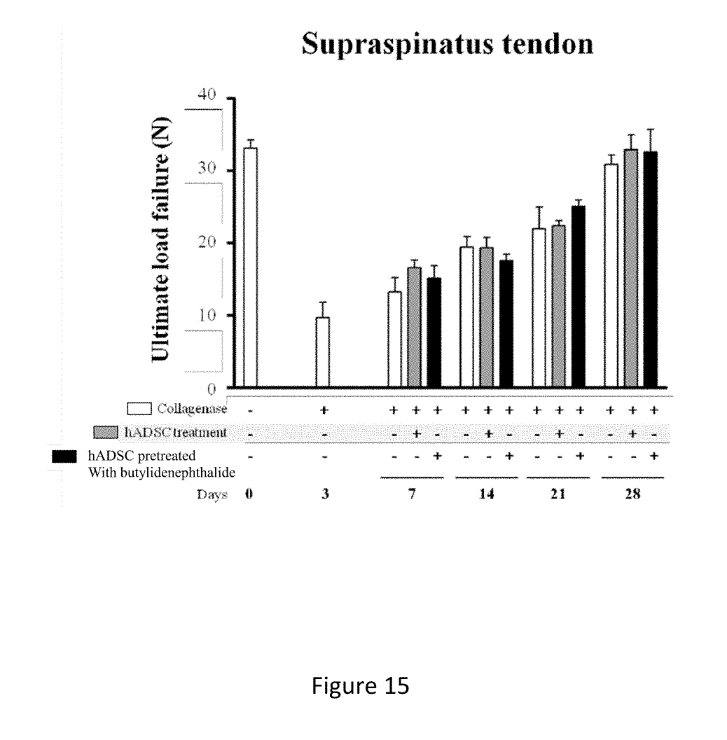

Referring to FIG. 15, the tensile strength of the supraspinatus tendon on day 3, 7, 14, 21, and 28 after treatment of type II collagenase was 9.71±6.63N, 13.26±4.34N, 19.46±3.59N, 21.99±7.39N, and 30.88±3.68N, separately. The tensile strength of normal tendon was 33.11±2.78N.

On day 3 after injection of collagenase, the hADSCs and pretreated hADSCs were injected to mice, respectively. On day 7, the tensile strength of the tendons treated with hADSCs was 15.24±4.29N, and the tensile strength of the tendons was 15.16±3.88N after injection of pretreated hADSc. The tensile strength of these two groups was higher than that of tendons in the untreated group. On day 14, the tensile strength of tendons treated with hADSCs and pretreated hADSCs was 19.36±3.19N and 17.47±2.21N, respectively. The tensile strength of these two groups was lower than that of untreated tendons. On day 21, the tensile strength of tendons was 25.11±1.77 after injection of pretreated hADSc, and the tensile strength of tendons treated with hADSCs was 22.41±1.76N. Compared to untreated group, the tensile strength of these two groups was higher. On day 28, the tensile strength of tendons treated with hADSCs and pretreated hADSCs was higher than control group. As mentioned above, the hADSCs had the abilities to inhibit/reduce the inflammation and repair/heal tendons. However, the injured tendons were not repaired completely.

viii. Treatment of Tendon Injuries by Different Cell Density

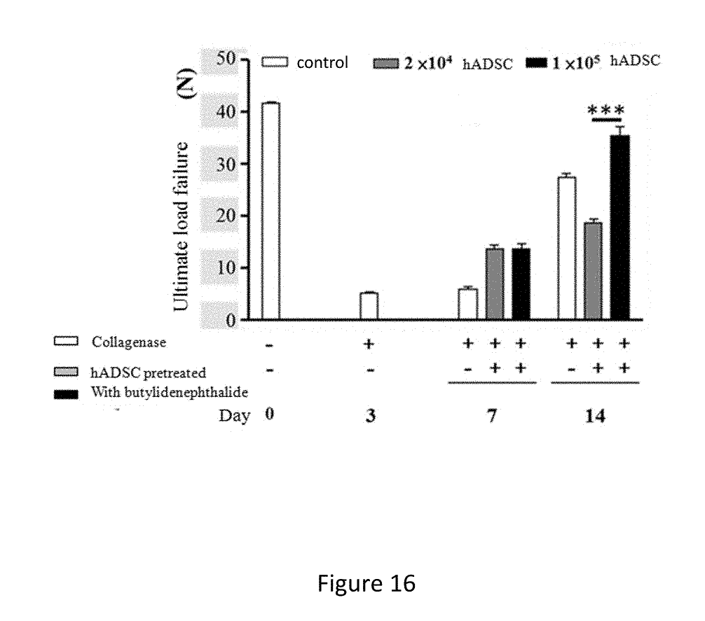

Referring to FIG. 16, after the mice were injected with 2.5 μg/ml butylidenephthalide pretreated hADSCs at different densities, 2×10⁴ or 1×10⁵ cell/ml, the restoration of tendon strength was different on day 7 and day 14.

ix. mRNA Expression of Tendon Healing Factors

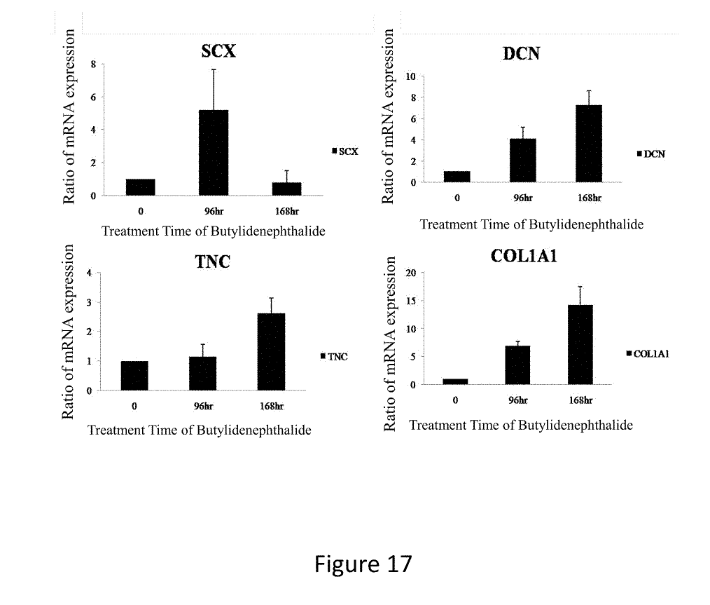

Referring to FIG. 17, after the hADSCs at a high cell density of 1×10⁵ cell/ml were treated with 2.5 μg/ml butylidenephthalide for 96 or 168 hours, the expression of COL1 protein (collagen, COL1A1) and its upstream gene (SCX) was increased, significantly.

x. Protein Expression of COL1A1

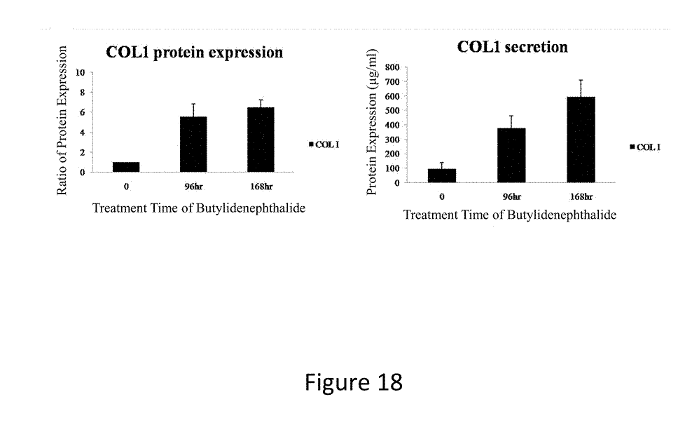

Referring to FIG. 18, after the hADSCs at a high cell density of 1×10⁵ cell/ml were treated with 2.5 μg/ml butylidenephthalide for 96 or 168 hours, the expression of COL1 protein (collagen, COL1A1) was increased, significantly.

TABLE 1
tissue grading of infraspinatus tendinitis in hADSCs treated and untreated groups

| | | Day 7 | | Day 14 | | Day 21 | | Day 28 | |
|---|---|---|---|---|---|---|---|---|---|
| Day 0 | Day 3 | vehicle | hADSC | vehicle | hADSC | vehicle | hADSC | vehicle | hADSC |
| n = 6 | n = 4 | n = 5 | n = 4 | n = 5 | n = 5 | n = 5 | n = 3 | n = 4 | n = 4 |

Tendon

| | | | | | | | | | | |
|---|---|---|---|---|---|---|---|---|---|---|
| Inflammation | − | −/+ | −/+ | −/+ | −/+ | −/+ | − | −/+ | − | − |
| Fiber arrangement | − | −/+ | −/+ | −/+ | −/+ | − | − | −/+ | − | − |
| Tenocyte structure | − | −/+ | + | −/+ | + | −/+ | − | −/+ | −/+ | −/+ |

Tendon and muscle middle

| | | | | | | | | | | |
|---|---|---|---|---|---|---|---|---|---|---|
| Inflammation | − | + | + | + | + | +/++ | −/+ | + | − | −/+ |
| Fiber arrangement | − | −/+ | + | −/+ | −/+ | − | − | −/+ | − | − |
| Tenocyte structure | − | −/+ | + | −/+ | + | −/+ | − | −/+ | −/+ | −/+ |

Muscle

| | | | | | | | | | | |
|---|---|---|---|---|---|---|---|---|---|---|
| Inflammation | − | + | + | −/+ | + | + | − | −/+ | − | −/+ |

TABLE 2
tissue grading of supraspinatus tendinitis in hADSCs treated and untreated groups

| | | Day 7 | | Day 14 | | Day 21 | | Day 28 | |
|---|---|---|---|---|---|---|---|---|---|
| Day 0 | Day 3 | vehicle | hADSC | vehicle | hADSC | vehicle | hADSC | vehicle | hADSC |
| n = 4 | n = 4 | n = 3 | n = 4 | n = 4 | n = 3 | n = 5 | n = 3 | n = 3 | n = 4 |

Tendon

| | | | | | | | | | | |
|---|---|---|---|---|---|---|---|---|---|---|
| Inflammation | − | ++/+++ | +++ | +/++ | ++ | + | +/++ | +/++ | + | −/+ |
| Fiber arrangement | − | ++/+++ | +++ | +/++ | +/++ | + | +/++ | + | ++/+++ | −/+ |
| Tenocyte structure | − | ++/+++ | +++ | +/++ | +/++ | +/++ | +/++ | −/+ | +/++ | −/+ |

Tendon and muscle middle

| | | | | | | | | | | |
|---|---|---|---|---|---|---|---|---|---|---|
| Inflammation | − | ++ | +++ | ++/+++ | ++/+++ | + | +/++ | + | + | − |
| Fiber arrangement | − | ++/+++ | ++/+++ | ++ | +/++ | +/++ | +/++ | −/+ | +/++ | −/+ |
| Tenocyte structure | − | ++/+++ | ++/+++ | ++ | ++ | +/++ | +/++ | −/+ | +/++ | −/+ |

Muscle

| | | | | | | | | | | |
|---|---|---|---|---|---|---|---|---|---|---|
| Inflammation | − | +/++ | + | +/++ | + | −/+ | + | −/+ | − | − |

TABLE 3
tissue grading of infraspinatus tendinitis in hADSCs treated (butylidenephthalide pretreatment) and untreated groups

| | | Day 7 | | Day 14 | | Day 21 | | Day 28 | |
|---|---|---|---|---|---|---|---|---|---|
| Day 0 | Day 3 | vehicle | hADSC | vehicle | hADSC | vehicle | hADSC | vehicle | hADSC |
| n = 6 | n = 4 | n = 5 | n = 6 | n = 5 | n = 3 | n = 5 | n = 6 | n = 4 | n = 4 |

Tendon

| | | | | | | | | | | |
|---|---|---|---|---|---|---|---|---|---|---|
| Inflammation | − | −/+ | −/+ | − | −/+ | −/+ | − | − | − | − |
| Fiber arrangement | − | −/+ | −/+ | − | −/+ | −/+ | − | −/+ | − | −/+ |
| Tenocyte structure | − | −/+ | + | − | + | −/+ | − | −/+ | −/+ | −/+ |

Tendon and muscle middle

| | | | | | | | | | | |
|---|---|---|---|---|---|---|---|---|---|---|
| Inflammation | − | + | + | − | + | −/+ | −/+ | − | − | − |
| Fiber arrangement | − | −/+ | + | − | −/+ | −/+ | − | −/+ | − | −/+ |
| Tenocyte structure | − | −/+ | + | −/+ | + | −/+ | − | −/+ | −/+ | −/+ |

Muscle

| | | | | | | | | | | |
|---|---|---|---|---|---|---|---|---|---|---|
| Inflammation | − | + | + | −/+ | + | − | − | − | − | − |

TABLE 4
tissue grading of supraspinatus tendinitis in hADSCs treated
(butylidenephthalide pretreatment) and untreated groups

| | | Day 7 | | Day 14 | | Day 21 | | Day 28 | |
|---|---|---|---|---|---|---|---|---|---|
| Day 0 | Day 3 | vehicle | hADSC | vehicle | hADSC | vehicle | hADSC | vehicle | hADSC |
| n = 4 | n = 4 | n = 3 | n = 6 | n = 4 | n = 4 | n = 5 | n = 5 | n = 3 | n = 6 |

Tendon

| | | | | | | | | | | |
|---|---|---|---|---|---|---|---|---|---|---|
| Inflammation | − | ++/+++ | +++ | ++/+++ | ++ | ++ | +/++ | −/+ | + | −/+ |
| Fiber arrangement | − | ++/+++ | +++ | ++/+++ | +/++ | ++ | +/++ | +/++ | ++/+++ | −/+ |
| Tenocyte structure | − | ++/+++ | +++ | ++ | +/++ | + | +/++ | + | +/++ | −/+ |

Tendon and muscle middle

| | | | | | | | | | | |
|---|---|---|---|---|---|---|---|---|---|---|
| Inflammation | − | ++ | +++ | +++ | ++/+++ | ++ | +/++ | −/+ | + | − |
| Fiber arrangement | − | ++/+++ | ++/+++ | ++/+++ | +/++ | ++ | +/++ | +/++ | +/++ | −/+ |
| Tenocyte structure | − | +/+++ | +/+++ | ++/+++ | ++ | ++ | +/++ | +/++ | +/++ | −/+ |

Muscle

| | | | | | | | | | | |
|---|---|---|---|---|---|---|---|---|---|---|
| Inflammation | − | +/++ | + | − | + | −/+ | + | − | + | − |

1. A composition for treating tendonitis comprising a pre-treated adipose derived stem cell, wherein the pre-treated adipose derived stem cell is pre-treated by a butylidenephthalide.

2. The composition of claim 1, wherein the pre-treated adipose derived stem cell express the SCX, DCN, TNC or COL1A1 marker on the cell.

3. The composition of claim 1, wherein a protein secreted by the pre-treated adipose derived stem cell is COL1.

4. The composition of claim 4, wherein the concentration of the butylidenephthalide is 2.5 to 5 μg/ml.

5. A method for manufacturing the pre-treating adipose derived stem cell of claim 1, comprising:
providing a adipose derived stem cell;
culturing the adipose derived stem cell in a medium containing butylidenephthalide; and
culturing the adipose derived stem cell in the medium for 96 to 168 hours.

6. The method of claim 5, wherein the concentration of butylidenephthalide is 2.5 to 5 μg/ml.

7. A method for treating tendonitis in a subject, wherein the method comprising:
administering a pharmaceutical composition as claim 1 to said subject.

8. The method of claim 7, wherein the cell number of the pre-treated adipose derived stem cell is 1×10⁵ to 3×10⁸ cells/ml.

9. The method of claim 7, wherein the tendonitis includes an infraspinatus tendinitis and/or a supraspinatus tendinitis.

10. The method of claim 9, wherein the infraspinatus tendinitis means the infraspinatus tendon and muscle middle is suffer from inflammation.

11. The method of claim 9, wherein the infraspinatus tendinitis means the infraspinatus tendon is suffer from tenocyte structure damage.

12. The method of claim 9, wherein the infraspinatus tendinitis means the infraspinatus muscle is suffer from inflammation.

13. The method of claim 9, wherein the supraspinatus tendinitis means the supraspinatus muscle is suffer from inflammation.

14. The method of claim 9, wherein the supraspinatus tendinitis means the supraspinatus tendon is suffer from fiber arrangement.

15. The method of claim 9, wherein the supraspinatus tendinitis means the supraspinatus tendon and muscle middle is suffer from inflammation.

16. The method of claim 7, wherein the administering is via injection.

17. The method of claim 16, wherein the injection is on day 3.
